(19) United States
(12) Patent Application Publication
(10) Pub. No.: US 2026/0240213 A1
(43) Pub. Date: Aug. 20, 2026

(54) HIGH LOAD HYDROGEL COMPOSITIONS

(71) Applicant: SOCIETE DES PRODUITS NESTLE S.A., VEVEY (CH)

(72) Inventor: Vesselin Danailov MILADINOV, Denville, NJ (US)

(21) Appl. No.: 19/532,253

(22) Filed: Feb. 6, 2026

Related U.S. Application Data
(60) Provisional application No. 63/760,744, filed on Feb. 20, 2025.

(51) Int. Cl. A23L 29/231; A23J 3/10; A23J 3/14; A23L 29/256; A23L 33/16; A23L 33/175; A23L 33/18; A23L 33/185; A23L 33/19

(52) U.S. Cl. CPC A23L 29/231; A23J 3/10; A23J 3/14; A23L 29/256; A23L 33/16; A23L 33/175; A23L 33/18; A23L 33/185; A23L 33/19

(57) ABSTRACT

The present invention provides an orally administrable hydrogel composition in a unit dosage form, containing: one or more protein with an isoelectric point of 4.5 or greater; and/or one or more cationic mineral salt; as well as carrageenan and/or low methoxyl amidated pectin; and the composition has a pH in the range of 3.8 to 5.0. The present invention also provides shelf-stable packet or container comprising one or more of the orally administrable hydrogel compositions and a method of manufacturing an orally administrable hydrogel composition in a unit dosage form.

### CROSS REFERENCE TO RELATED APPLICATIONS

This application claims priority to U.S. Provisional Application No. 63/760,744 filed Feb. 20, 2025, the entire contents of which are incorporated herein by reference.

### FIELD OF THE INVENTION

The present invention relates to orally administrable hydrogel compositions in a unit dosage form. In particular, the present invention relates to orally administrable hydrogel compositions with mildly acidic pH comprising high amounts of proteins and/or cationic mineral salts, and carrageenan and/or low methoxyl (LM) amidated pectin as gelling agents.

### BACKGROUND

Nutraceutical compositions are a common way to fortify the diet of healthy individuals, those predisposed to disease and those suffering from disease. Examples of active nutraceutical ingredients include proteins, fats, vitamins, minerals and micronutrients. Generally, active nutraceutical ingredients are given at a constant dosage, often on a daily basis.

For example, the European Food Safety Authority (EFSA) recommends Population Reference Intakes (PRIs) of calcium of between 400 and 1150 mg/day for a child, 1000 mg/day for young adults (18-24 years) and 950 mg/day for adults ≥25 years (EFSA Journal 2015, 13, 4101). For magnesium, the EFSA calculated Adequate Intakes (AIs) of 170 mg/day for children aged 1-3 years, 230 mg/day for children aged 3-10, 250-300 mg/day for children aged 10-18 and 250-300 mg/day for adults.

Likewise, function proteins often need to be consumed in gram dosages. For example, studies suggest that the effective amounts of functional collagen peptides can be 2.5 to 15 g/day (Paul C., et al. Nutrients 2019, 11, 1079; and references cited therein).

These high-milligram to low-gram dosages are amenable to administering in unit dosages, such as tablets, capsules or dragées. This ensures that the correct dosage is taken in a way that is convenient to the consumer. Such unit dosage forms can provide gram quantities of nutrients with only a small number of unit dosages need to be taken each day. However, such formulations can be difficult to swallow and may be perceived by consumers as medicines rather than a dietary supplement.

One solution to this problem is formulating nutraceutical compositions as unit dosage hydrogels that resemble sweets or candies and are known as “gummies”. This ensures that the unit dosages can be chewed, if required, and are perceived by consumers as nutraceutical supplements rather than medicines. However, current gummies are limited in the amount of protein (e.g., animal proteins, dairy proteins or plant proteins) and minerals (e.g., calcium and/or magnesium) they can carry per gram without adversely affecting the taste or requiring high amounts of stabilisers. Unit dosages are limited to weights of ~5 g.

WO 2021/016665 discloses gummy compositions with protein (collagen) and a gelling agent (gelatine). The final product contains 24-28% by weight of protein (~20 wt % collagen and 8 wt % gelatine) and product was described as having “a pleasant taste and texture”. However, no stability data were provided, which is important to ensure that the gummies can be packaged (e.g., in tubs) without sticking or melting.

US 2022/0062350 discloses oil-in-water emulsion gummy compositions with ~25% by weight calcium and/or magnesium. However, their stability is variable (even at only 45° C.), and they require calorific fats and emulsifiers in high amounts (e.g., 20 wt % or more) to achieve such high concentrations of minerals. Whilst fats (e.g., omega-3 fats) may form part of nutraceutical compositions, it may not be desirable that they are required in high amounts because many individuals do not require fat supplementation.

JP5429033B2 discloses a gummy with 50 to 80 wt % collagen, 5 to 10 wt % fat and 12 to 36% glycerol that are substantially free of sugar and starch syrup. The pH, taste properties and stability are not disclosed. Large amounts of glycerol may not be recommended for some consumers, such as young children.

Furthermore, gummies typically have slightly sour taste, usually achieved at mildly acidic pH values (e.g., a pH of about 5 or less). When large amounts of active ingredients such as proteins (e.g., collagen) and/or cationic mineral (e.g., calcium and magnesium) are added, a large amount of acid may be required to titrate the basic active ingredients. When large amounts of acid are added, even a pH between 4 and 5, the flavour is intensely sour. The higher the load of these active ingredients, the higher the pH needs to be to have acceptable taste.

Therefore, there remains a need to provide unit dosage forms with high-milligram to low-gram dosages of proteins and/or minerals that are perceived as supplements rather than medicines and which have acceptable taste and shelf stability. Furthermore, the unit dosage forms should be stable to moderately elevated temperatures (e.g., 45 or 68° C.) as an indication that they can be stored at ambient temperature (e.g., 25° C.) for extended periods (i.e., shelf stable for 1 year). This balance of properties should be achievable without the need for large quantities of stabilisers (e.g., glycerol, fats or emulsifiers) that might limit the usability of a nutraceutical unit dosage in certain individuals.

### SUMMARY

Surprisingly, it has been discovered that hydrogels with high amounts of protein (e.g., animal proteins, dairy proteins and/or plant proteins) and/or minerals (e.g., calcium and/or magnesium) can be produced at mildly acidic pH (e.g., a pH of about 5 or less) when low methoxyl (LM) amidated pectin and/or carrageenan are used as gelling agents. The hydrogels do not require high amounts of fats or glycerol to stabilise them, nor are they intensely sour, yet they remain stable to moderately elevated temperatures (e.g., 45 or 68° C.). In particular, it has been discovered that cationic mineral salts and proteins with an isoelectric point (pI) of 4.5 or greater (i.e., that maintain some positively charge close to and below the pI) form stable, palatable “gummy” formulations in the presence of LM amidated pectin and/or carrageenan.

In a first general embodiment, the present invention provides an orally administrable hydrogel composition in a unit dosage form, comprising: 15 to 50 wt % of one or more protein with an isoelectric point of 4.5 or greater; and/or 8 to 50 wt % of one or more cationic mineral salt; as well as 1 to 5 wt % carrageenan and/or low methoxyl amidated pectin (which may act as a gelling agent); wherein the composition has a pH in the range of 3.8 to 5.0. The amounts of the components in wt % are based on the total weight of the hydrogel composition including the water (e.g., excluding any coating such as oil/wax or sugar or coatings).

In a second general embodiment, the present invention provides a shelf-stable packet or container comprising one or more of the orally administrable hydrogel compositions according to the first embodiment.

In a third general embodiment, the present invention provides a method of manufacturing an orally administrable hydrogel composition in a unit dosage form, for example, the orally administrable hydrogel composition of the first general embodiment.

### DETAILED DESCRIPTION

### Definitions

The term “hydrogel” is defined as a polymeric material with a three-dimensional porous network formed by the cross-linking (physically and/or chemically) of polymer chains, which retains water in its spatial structure. Water contents of 10 to 60 wt % may be employed with lower amounts of water (preferably 15 to 30 wt %, more preferably 18 to 25 wt %) preferred to improve shelf stability. Beyond the requirement for water, hydrogels require a “gelling agent” to induce gelation (e.g., 1 to 5 wt % low methoxyl amidated pectin and/or carrageenan). To be “orally administrable”, the ingredients must be edible, e.g., consumable by a human. Edible gelling agents are known to the skilled person and include gelatine (a proteinaceous gelling agent produced by partially hydrolysing collagen) and carbohydrate gelling agents such as agar, low methoxyl (LM) amidated pectin, high methoxyl (HM) pectin, carrageenan and gellan gum. Once hydrogels are moulded into a unit dosage form (e.g., 0.5 to 5 g) they are often referred to as “gummies”.

The term “pectin” is a heteropolysaccharide extracted from terrestrial plants. Pectin has a large concentration of galacturonic acid carboxyl groups, which results in a net negative charge in the neutral and mildly acidic pH range. Pectin with more than 50% of the galacturonic acid carboxyl groups esterified with methanol is referred to as “high methoxyl pectin” or “HM pectin”. Pectin with 5 to 50% of its carboxyl groups esterified with methanol is referred to as “low methoxyl pectin” or “LM pectin”. Pectin may be amidated with ammonia to form carboxylic acid amides when it is known as “amidated pectin”.

The term “low methoxyl amidated pectin” or “LM amidated pectin” refers to partially amidated pectin with 5 to 50% of its carboxyl groups esterified with methanol.

The term “carrageenan” refers to a family of natural linear sulfated polysaccharides extractable from red seaweeds. It generally has a negative charge in the range 3.8 to 5.0.

The term “gelatine” refers to a denatured form of collagen that is partially hydrolysed by acid or base. It is not contemplated that gelatine will be a primary active ingredient because it is generally used as a gelling agent in amounts less than 15 wt %. However, gelatine may contribute to the total protein content. Gelatine may have a molecular weight of about starting about 10 kDa and ending at about 400 kDa with a major part of the molecular weight fraction have a molecular weight around 100 kDa, which correlates with the propensity to gel Preferred gelatines for gummies have a Bloom strength of 150-325 (e.g., average molecular masses of 40 to 100 kDa; e.g., Gelatine type A or B), more preferably a Bloom strength of 225-325 (e.g., average molecular masses of 50 to 100 kDa; e.g., Gelatine type A). In contrast, collagen hydrolysates or collagen peptides, which do not readily gel, have molecular weights starting about 0.5 kDa and ending at about 25 kDa with a major part of the molecular weight fraction having a molecular weight around 1 to 10 kDa.

The term “isoelectric point” or “pI” of a protein or peptide is the pH value at which point protein has a net charge of zero. The pI value of most dietary proteins and peptides is known to those skilled in the art. The pI value for a given protein may be determined using gel-based isoelectric focusing (IEF) or capillary isoelectric focusing (cIEF) methods.

The term “protein with an isoelectric point of 4.5 or greater” refers to proteins or peptides which have a net charge of zero at pH 4.5 or greater, so will have a net positive charge throughout most or all of the pH range from 3.8 to 5.0. Specific examples of such proteins include dairy proteins (e.g., casein (pI≈4.6) or whey protein (pI 4.6-5.2)), other animal derived proteins (e.g., collagen (pI=4.5-9) or lysozyme (pI=10-11)) and many plant proteins (e.g., soy proteins or pea protein isolate (pI≈4.5)). Most collagen peptides have an average pI of 4.5 or greater.

The term “collagen” refers to the main structural protein in the extracellular matrix of the connective tissues of many animals that has not been actively denatured or hydrolysed prior to its use as an ingredient in the gummy compositions. Type A collagen has an isoelectric point (pI) of 8-9 and type B collagen has an isoelectric point (pI) of 4-5. Non-hydrolysed collagen has a molecular weight of about 100-300 kDa. Collagen may be hydrolysed to collagen peptides with increased bioavailability, but which to not act as gelling agents. The person skilled in the art can distinguish collagen from gelatine, which is included to cause gelation. The term “hydrolysed collagen”, “collagen peptides” or “a hydrolysed version of collagen” refers to a hydrolysed form of collagen that that does not induce gelation independently at a concentration of 1 to 5 wt %. Collagen peptides produced by alkaline hydrolysis generally have isoelectric points between 4.5 and 6.5 (e.g., PP XB (beef hide)=5.69; PP XBO (bovine bone)=5.14; PP XP (pig skin)=6.27).

The term “cationic mineral salt” refers to nutritional minerals that can form cationic salts (e.g., calcium, magnesium, iron, zinc, chromium, copper, molybdenum, manganese, nickel, selenium or vanadium). Suitable water-soluble salts for use in hydrogels are known to those skilled in the art and should generally have solubility of at least 2 wt %, preferably at least 5 wt %, more preferably at least 8 wt % in water (pH 3.8 to 5.0). Preferred counterions include those that stabilise or enhance the solubility of the cationic mineral (e.g., ascorbate, citrate, lactate, fumarate, tartrate, maleate, gluconate and/or an amino acid and/or a vitamin (i.e., pantothenate)). Where the counterion is multidentate, it may form a chelate salt with the cationic mineral. This is particularly the case with polyacids or amino acids. Preferred amino acids include the 20 canonical amino acids (i.e., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine). It is understood that the counterions are excluded from the definition of protein (above) or organic acid (below). When discussing amounts of “cationic mineral salt” in wt %, this includes the weight of the metal and associated counterion.

The term “organic acid” is a standard term of the art including ascorbic acid, citric acid, lactic acid, fumaric acid, tartaric acid, malic acid. Where the claims further comprise “one or more organic acid” it is understood that this excludes any salt forms added as part of the cationic mineral salt (e.g., calcium or magnesium: ascorbate, citrate, lactate, fumarate, tartrate, maleate or gluconate).

The term “bulking agent” is an optional solid carrier that provides volume to the unit dosage form without requiring additional water. For example, suitable bulking agents include carbohydrate bulking agents such as a sugar, a maltodextrin (including corn syrups), a sugar alcohol and/or a fibre.

The term “a fatty component” refers to a fat (i.e., a triglyceride), one of its fatty component parts (e.g., a diglyceride, monoglyceride or fatty acid) or an emulsifier. Glycerol in its free form is not considered a fatty component because it is hydrophilic.

The term “shelf stable” is defined as the unit dosage form retaining its shape for at least 6 months in a container at 25° C. and 50% humidity.

The term “weight %” or “wt %” refers to the amount of a component or type of component present in the orally administrable hydrogel composition in a unit dosage form and includes any water present within the composition. This does not include any coating, such as an oil/wax or sugar coating, that does not form part of the hydrogel.

The term “about” means±10% of a given value, for example, 10 includes rounding errors of 9.5 to 10.499, whereas about 10 includes 9 to 11.

Unless expressly stated otherwise, all numerical values and numerical ranges disclosed herein are intended to be understood as inherently approximated and as explicitly supporting every conceivable sub-range, sub-set, and individual value therein. Each disclosed range shall be construed to include its endpoints and all integers, whole numbers, fractional numbers, and decimal values encompassed between those endpoints. For the avoidance of doubt, disclosure of any numerical range, whether expressed as ‘from X to Y,’ ‘between X and Y,’ or otherwise, provides written description support for claims directed to: (i) any narrower range wholly contained within the broader range; (ii) any range having endpoints selected from among the values within the disclosed range; and (iii) any specific value, integer, whole number, fractional value, or decimal value within that range. For example, disclosure of a range of 1 to 10 should be interpreted as providing support for any and all sub-ranges, such as 1-8, 3-7, 1-9, 3.6-4.6, 3.5-9.9, or any other sub-range or specific value contained therein. Each disclosed range also supports claims reciting open-ended or closed-ended ranges sharing at least one boundary with the disclosed range. The foregoing principles apply regardless of whether numerical values are presented explicitly, graphically, or through examples, and regardless of the precision of the stated values.

### An Orally Administrable Hydrogel Composition in a Unit Dosage Form

In a first general embodiment, the present invention provides an orally administrable hydrogel composition in a unit dosage form, comprising:
15 to 50 wt % of one or more protein with an isoelectric point of 4.5 or greater; and/or 8 to 50 wt % of one or more cationic mineral salt; and
1 to 5 wt % low methoxyl amidated pectin and/or carrageenan (which may act as a gelling agent);
wherein the composition has a pH in the range of 3.8 to 5.0;
wherein the amounts in wt % are based on the total weight of the hydrogel composition (e.g., excluding any coating such as oil/wax or sugar coatings).

This first general embodiment provides “gummies” with a high load of proteins and/or cationic mineral salts with acceptable taste and good stability.

### Primary Active Nutraceutical Ingredients-Proteins and Minerals

Proteins with an Isoelectric Point of 4.5 or Greater

The primary ingredient may be a protein with an isoelectric point of 4.5 or greater. The protein with an isoelectric point of 4.5 or greater is not particularly limited.

The protein with an isoelectric point of 4.5 or greater may be an animal protein or a hydrolysed version thereof. Animal proteins can be rich in essential amino acids and provide benefits to the hair, skin or joints. Sources of animal protein include collagen (pI=4.5-9) or lysozyme (pI=10-11). A preferred animal protein is collagen. The collagen may be intact or hydrolysed collagen, preferably hydrolysed collagen (e.g., collagen peptides). As defined above, collagen peptides do not readily gel at concentrations of 1 to 5 wt % and can be readily distinguished from gelatine. In an embodiment, the protein with an isoelectric point of 4.5 or greater is not gelatine.

The protein with an isoelectric point of 4.5 or greater may be a diary protein (e.g., a whey protein, a casein protein, a hydrolysed version thereof, a hydrolysed version thereof, or any combination thereof, for example, milk protein with a casein: whey ratio of about 80:20). Dairy proteins are rich sources of amino acids and are readily available. Sources of dairy protein include skimmed milk powder, milk protein concentrate, milk protein isolate, whey protein concentrate, whey protein isolate, micellar casein concentrate, micellar casein isolate, acid caseinate, sodium caseinate, potassium caseinate, magnesium caseinate or calcium caseinate. A preferred dairy protein is whey protein (pI 4.6-5.2), more preferably whey protein isolate.

The protein with an isoelectric point of 4.5 or greater may be a plant protein. Plant proteins are suitable for vegetarians and vegans, or those who do not eat certain animal products from pigs or cows. Sources of plant proteins include pea protein (pI≈4.5), soy protein (pI≈4.5), rapeseed protein (pI=7-11) or a hydrolysed version thereof. A preferred plant protein is pea protein or a hydrolysed version thereof.

Therefore, preferred proteins with an isoelectric point of 4.5 or greater are selected from the group consisting of collagen, lysozyme, milk protein, casein, whey protein, pea protein, soy protein, rapeseed protein, a hydrolysed version thereof, and any mixture thereof. A particularly preferred protein is hydrolysed collagen (e.g., collagen peptides).

The protein may be hydrolysed or non-hydrolysed. Preferably the protein is hydrolysed. Hydrolysed proteins may have improved bioavailability and reduce viscosity.

The one or more protein with an isoelectric point of 4.5 or greater is present in an amount of 15 to 50 wt %, preferably 18 to 40 wt %, more preferably 20 to 35 wt %, even more preferably 20 to 30 wt %; wherein the amounts in wt % are based on the total weight of the hydrogel composition (e.g., excluding any coating such as oil/wax or sugar coating). The unit dosage may contain a single type of protein as the primary active (protein) ingredient. For example, one or more animal protein (e.g., collagen or lysozyme) or a hydrolysed version thereof may be present in an amount of 15 to 50 wt %, preferably 18 to 40 wt %, more preferably 20 to 30 wt %. For example, one or more diary protein (e.g., a whey protein, a casein protein or a combination thereof, for example, milk protein with a casein: whey ratio of about 80:20) or a hydrolysed version thereof may be present in an amount of 15 to 50 wt %, preferably 15 to 40 wt %, more preferably 15 to 30 wt %, even more preferably 20 to 30 wt %. For example, one or more plant protein (e.g., pea protein, soy protein or rapeseed protein) or a hydrolysed version thereof may be present in an amount of 15 to 50 wt %, preferably 18 to 40 wt %, more preferably 20 to 30 wt %, even more preferably 20 to 30 wt %. In an embodiment, the amount of protein with an isoelectric point of 4.5 or greater does not include any gelatine. Gelatine is generally included in amounts of less than 15 wt %, preferably less than 10 wt %, more preferably less than 5 wt %, most preferably less than 1 wt %.

### Cationic Mineral Salts

The primary ingredient may be a cationic mineral salt. The cationic mineral salt is more limited because only certain cationic mineral salts are used in nutritional compositions including calcium, magnesium, iron, zinc, chromium, copper, molybdenum, manganese or selenium. Preferred cationic mineral salts are selected from the group consisting of calcium, magnesium and/or zinc, more preferably calcium and/or magnesium. Calcium and magnesium are required in the largest daily dosages (e.g., more than 100 mg/day) with zinc being required at intermediate daily dosages (~10 mg/day).

The counterion for the cationic mineral salt is not particularly limited. Preferred counterions include ascorbate, citrate, lactate, fumarate, tartrate, maleate, gluconate, acetate, and/or an amino acid and/or vitamin (i.e., pantothenate).

Preferred amino acids are alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. More preferred cationic mineral salts include calcium ascorbate, calcium citrate, magnesium glycinate and magnesium citrate.

A preferred vitamin is pantothenate, preferably calcium-d-pantothenate.

The one or more cationic mineral salt is present in an amount of 8 to 50 wt %, preferably 10 to 40 wt %, more preferably 12 to 25 wt %, even more preferably 15 to 25 wt %; wherein the amounts in wt % are based on the total weight of the hydrogel composition (e.g., excluding any oil/wax or sugar coating). The unit dosage may contain a single type of cationic mineral salt as the primary active (cationic mineral) ingredient. For example, one or more calcium salt may be present in an amount of 8 to 50 wt %, preferably 10 to 40 wt %, more preferably 12 to 25 wt %, even more preferably 15 to 25 wt %. For example, one or more magnesium salt may be present in an amount of 8 to 50 wt %, preferably 8 to 40 wt %, more preferably 8 to 25 wt %, even more preferably 15 to 25 wt %. The unit dosage may contain a combination of cationic mineral salts as the primary active (cationic mineral) ingredient. For example, a combination of calcium, magnesium and zinc salts may be present in an amount of 8 to 50 wt %, preferably 10 to 40 wt %, more preferably 12 to 25 wt %, even more preferably 15 to 25 wt %. For example, a combination of calcium and magnesium salts may be present in an amount of 8 to 50 wt %, preferably 10 to 40 wt %, more preferably 12 to 25 wt %, even more preferably 15 to 25 wt %. As cationic mineral salts other than calcium, magnesium and zinc are generally required in microgram dosages, these generally will not form the primary active ingredient, but may be present alongside calcium, magnesium and/or zinc. Such microgram (trace) dosages are unlikely to contribute more than 0.5 wt % of the composition.

Combinations of Proteins with an Isoelectric Point of 4.5 or Greater and Cationic Mineral Salts

It will be clear to those skilled in the art that the disclosed amounts of the protein with an isoelectric point of 4.5 or greater and cationic mineral salt may be combined into a single orally administrable hydrogel composition in a unit dosage form. In this case, the total amounts of these components (in weight %) will be limited by the need for an amount of pectin and/or carrageenan and an amount of water. Therefore, where the hydrogel composition comprises one or more protein with an isoelectric point of 4.5 or greater and one or more cationic mineral salt, the total amount of these components (i.e., the primary active ingredients) may not exceed 87 wt % (e.g., to accommodate 1% gelling agent and 12 wt % water), preferably not exceed 82 wt % (e.g., to accommodate 5% gelling agent and 10 wt % water), more preferably not exceed 75 wt % (e.g., to accommodate significant amounts of water and/or bulking agents), even more preferably not exceed 55 wt % (e.g., to accommodate significant amounts of water and/or bulking agents); wherein the amounts in wt % are based on the total weight of the hydrogel composition (e.g., excluding any coating such as oil/wax or sugar coating). For example, when both types of primary active ingredient are present, an amount of protein with an isoelectric point of 4.5 plus cationic mineral salt may be 23 to 87 wt %, preferably 23 to 80 wt %, more preferably 23 to 55 wt %, even more preferably 35 to 55 wt %.

Gelling Agents—Carrageenan and/or Low Methoxyl Amidated Pectin

The orally administrable hydrogel composition in a unit dosage form comprises a gelling agent selected from the group consisting of carrageenan and low methoxyl amidated pectin. These gelling agents facilitate the formation of hydrogel compositions with high amounts of protein (e.g., animal proteins, dairy proteins and/or plant proteins) and/or minerals (e.g., calcium and/or magnesium) that can be produced at mildly acidic pH.

Preferred low methoxyl amidated pectins include those with 20-40% methoxylated pectins and 10-25% amidation (e.g., LM-101AS from CP Kelco (Chicago, IL, USA)).

Preferred carrageenans include t-carrageenan and/or K-carrageenan. In particular, k-carrageenan is preferred when combined with dairy proteins and 1-carrageenan is preferred when combined with metal ions. Many carrageenans are known to those skilled in the art (McHugh D, FAO Fisheries Technical Paper 441, Chapter 7, 2003).

The carrageenan and/or low methoxyl amidated pectin is present in an amount of 1 to 5 wt %, preferably 1.4 to 3 wt %, more preferably 1.6 to 2.8 wt %; wherein the amounts in wt % are based on the total weight of the hydrogel composition (e.g., excluding any coatings such as oil/wax or sugar coating).

pH

The orally administrable hydrogel composition in a unit dosage form has a pH in the range of 3.8 to 5.0, preferably in the range of 3.8 to 4.5, more preferably in the range of 4.0 to 4.5.

### Optional Ingredients

The orally administrable hydrogel composition in a unit dosage form may include one or more optional ingredients as outlined below. Such optional ingredients may be combined without limitation.

### One or More Organic Acid

The orally administrable hydrogel composition in a unit dosage form may include one or more organic acid. Organic acids are included to when larger amounts of active ingredients such as proteins (e.g., collagen) and/or cationic mineral are added (e.g., calcium and magnesium) to titrate any basic active. The amount of acid is not particularly limited and will depend on the amount and basicity of the primary active ingredients, the strength and concentration of the acids and the final pH of the product. Organic acid may not be required when acidic ingredients are used, e.g., acid casein or acidic bulking agents such as sugar alcohols. When added, the one or more organic acid may be present in an amount of 0.1 to 20 wt %, preferably 1.0 to 15 wt %; wherein the amounts in wt % are based on the total weight of the hydrogel composition (e.g., excluding any oil/wax or sugar coating).

The identity of the organic acid is not particularly limited and many organic acids are known to those skilled in the art. Preferred organic acids may be selected from the selected from the group consisting of ascorbic acid, citric acid, lactic acid, fumaric acid, tartaric acid, acetic acid, gluconic acid, and/or malic acid, more preferably citric acid, lactic acid and/or ascorbic acid even more preferably citric acid and/or lactic acid. Where a specific metal ion chelator is included (e.g., citrate or ascorbate) it may be preferably to use the equivalent organic acid (e.g., citric acid or ascorbic acid).

### One or More Bulking Agent

The orally administrable hydrogel composition in a unit dosage form may include one or more bulking agent. Bulking agents are included when the highest amounts of primary active ingredients are not required yet high amounts of water are not desired. The amount of bulking agent is not particularly limited and will depend on the amount of the primary active ingredients and the desired water content of the hydrogel composition. When added, the one or more bulking agent may be present in an amount in an amount of 1 to 81 wt %, 20 to 80 wt %, preferably 30 to 70 wt %, more preferably 40 to 60 wt %; wherein the amounts in wt % are based on the total weight of the hydrogel composition (e.g., excluding any coatings such as oil/wax or sugar coating).

The identity of the bulking agent is not particularly limited, and many bulking agents are known to those skilled in the art. Carbohydrate bulking agents are generally preferred. Preferred carbohydrate bulking agents may be selected from the selected from the group consisting of a sugar, a maltodextrin (e.g., corn syrup), a sugar alcohol (e.g., maltitol) and/or a fibre, preferably a sugar (e.g., sucrose) or a maltodextrin (e.g., corn syrup). The types of bulking agents are generally selected to ensure the required sweetness, so balance sweeter sugars with less sweet maltodextrins.

Preferred sugars include fructose, galactose, glucose, lactose, maltose or sucrose, more preferably sucrose. Preferred maltodextrins (e.g., a corn syrup) have a dextrose equivalent (DE) of from 2 to 73, more preferably 32 to 73, for example a corn syrup DE42 (e.g., Globe 42 DE Corn Syrup) or corn syrup DE63 (e.g., Globe 62 DE Corn Syrup), in particular a corn syrup DE42 (e.g., Globe 42 DE Corn Syrup).

Preferred sugar alcohols include those with 4 carbon atoms (tetrose), 5 carbon atoms (pentose) or 6 carbon atoms (hexose), more preferably erythritol, xylitol, isomalt, mannitol or sorbitol. More preferred sugar alcohols that are disaccharides, trisaccharides and oligosaccharides, even more preferably maltitol and/or hydrogenated starch hydrolysates. Glycerol (a sugar alcohol with 3 carbon atoms that is released from fats by hydrolysis) is not preferred in its free form because large quantities may not be recommended for young children. In a preferred embodiment, the hydrogel composition contains low levels of glycerol in a free form (e.g., less than 5 wt %, preferably less than 1 wt %, even more preferably the composition is substantially free or free from glycerol).

Preferred fibres include soluble dietary fibres, more preferably inulin, fructooligosaccharides, galactooligosaccharides corn fibre, and/or polydextrose.

More preferably, the bulking agent in the hydrogel composition comprises from 26 to 36 wt % sucrose, from 18 to 28 wt % 53 to 73 DE corn syrup and from 8 to 18 wt % 32 to 52 DE corn syrup, based on the total weight of the hydrogel composition. The 53 to 73 DE corn syrup is preferably about 63 DE corn syrup (such as Globe 63 DE Corn Syrup). The 32 to 42 DE corn syrup is preferably about 42 DE corn syrup (such as Globe 42 DE Corn Syrup).

### One or More Fatty Component

The orally administrable hydrogel composition in a unit dosage form may include one or more fatty component. Fatty components are included in gummies for two reasons: to provide nutrition or to stabilise the composition, for example, in the form of an oil-in-water emulsion (US 2022/0062350). The disclosed hydrogel compositions do not require a fatty component to stabilise the composition but may include a fatty component to provide nutrition.

When present, the one or more fatty component may be selected from the group consisting of a fat, a diglyceride, a monoglyceride and/or an emulsifier (e.g., a lecithin). In a preferred embodiment, the fatty component comprises one or more fat (i.e., triglycerides) and, optionally a small amount of one or more a diglyceride, a monoglyceride and/or an emulsifier.

The amount of fatty component is not particularly limited and will depend on the nutrient required. When added, the one or more fatty component may be present in an amount of 0.1 to 10 wt %, preferably 1 to 9 wt %, more preferably 2 to 8 wt %, even more preferably 3 to 7 wt %; wherein the amounts in wt % are based on the total weight of the hydrogel composition (e.g., excluding any oil/wax or sugar coating). It is contemplated that when the fatty component is present, the majority will be in the form of fat with 2 wt % or less provided by the diglyceride, a monoglyceride and/or an emulsifier. For example, one or more fats may be present in an amount of 0.1 to 9 wt %, preferably 1 to 8 wt %, more preferably 2 to 7 wt %, even more preferably 3 to 6 wt %.

The identity of the fatty component is not particularly limited and nutritional fats are known to those skilled in the art. Preferably, the fatty component comprises a fat such as a fat comprising polyunsaturated fatty acids, more preferably omega-3 fatty acids, even more preferably α-linolenic acid (18:3n-3), eicosapentaenoic acid (20:5n-3) and/or docosahexaenoic acid (22:6n-3). Alternatively, or additionally, the fatty component comprises a fat such as a fat comprising medium chain fatty acids, preferably hexanoic, octanoic and/or decanoic acid, more preferably octanoic and/or decanoic acid. Optionally, the fatty component comprises one of more emulsifier, preferably a lecithin, a monoglyceride and/or diglyceride (glycerides that act as emulsifiers due to the free alcohol on the glyceride “head” group and fatty acid “tail” group(s)).

In one embodiment, the composition contains less than 10 wt % of the fatty component, preferably less than 5 wt %, more preferably less than 1 wt %, even more preferably substantially free or free from a fat, a diglyceride, a monoglyceride and/or an emulsifier.

### One or More Additional Vitamin, Mineral or Micronutrient

The orally administrable hydrogel composition in a unit dosage form may include one or more additional vitamin, mineral or micronutrient beyond the primary active ingredients. For example, the unit dosage form may be a “multivitamin” enriched with the primary active ingredients disclosed above. For example, the unit dosage form may combine vitamins, minerals and micronutrients with a specific purpose (e.g., calcium plus vitamin D to improve bone health; collagen hydrolysate plus glucosamine, chondroitin, hyaluronic acid, oleuropein and/or hydroxytyrosol to improve skin, hair or cartilage; or calcium and whey protein plus vitamin D to improve muscle).

Suitable vitamins may be selected from the group consisting of vitamin A, vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid), and vitamin B12 (various cobalamins; commonly cyanocobalamin in vitamin supplements), vitamin C, vitamin D, vitamin E, vitamin K, choline, or combinations thereof.

Suitable (additional) minerals may be selected from the group consisting of boron, iodine, phosphorus, potassium, silicon, tin, or combinations thereof. Where the primary active ingredient is one or more cationic mineral salt, and this is limited to specific cationic metals (e.g., calcium, magnesium and/or zinc), the additional minerals may include small amounts (e.g., less than 1 wt %) of other cationic minerals such as chromium, copper, iron, manganese, molybdenum, nickel, selenium, vanadium, or combinations thereof.

Suitable (additional) micronutrients may be selected from the group consisting of phytonutrients (e.g., terpenoids (isoprenoids) such as carotenoids, triterpenoid, monoterpenes and steroids; phenolic compounds, for example natural monophenols, polyphenols (e.g. flavonoids, isoflavonoid, flavonolignan, lignans, curcuminoids, stilbenoid and hydrolysable tannin); aromatic acids (e.g. phenolic acids and hydroxycinnamic acids); capsaicin; phenylethanoids; alkylresorcinols; glucosinolates; betalains and chlorophylls), antioxidants (e.g., astaxanthin, carotenoids, coenzyme Q10 (“CoQ10”), flavonoids, glutathione Goji (wolfberry), hesperidin, lacto-wolfberry, lignan, lutein, lycopene, polyphenols and zeaxanthin), nucleotides, glucosamine, chondroitin, hyaluronic acid, oleuropein and/or hydroxytyrosol.

Preferably, the amount of vitamin E is less than 2 wt % of the composition, and the amounts of each other additional vitamin, mineral or micronutrient may optionally account for at least 1 wt % of the composition.

### Preferred Properties of the Orally Administrable Hydrogel Composition in a Unit Dosage Form

The hydrogel composition is in a unit dosage form and must be suitable for oral administration. Such a unit dosage form must fit in the mouth of an individual and may be swallowed whole or chewed. Preferably, the unit dosage form has a weight of 0.5 to 9 g, preferably 1 to 4 g, more preferably 2 to 3 g. This allows the hydrogel composition to be administered orally, optionally with chewing to accommodate large unit dosages.

As will be described below, the orally administrable hydrogel compositions are stable. Preferably, the hydrogel composition stable at 45° C. to 68° C., more preferably at 68° C., in closed containers for 3 hours, preferably for 24 hours. This ensures that the hydrogel compositions are shelf stable.

### Shelf-Stable Packet or Container Comprising One or More of the Orally Administrable Hydrogel Compositions

In a second general embodiment, the present invention provides a shelf-stable packet or container comprising one or more of the orally administrable hydrogel compositions according to the first embodiment.

The packet or container is preferably selected from the group consisting of a tube, a tub, a pouch, a sachet, a bottle or a blister pack. The packet or container optionally includes instructions for administration.

### Methods of Manufacturing Orally Administrable Hydrogel Compositions

In a third general embodiment, the present invention provides a method of manufacturing an orally administrable hydrogel composition in a unit dosage form, the method comprising the following step:
a) adding to water to form a mixture:
ai) low methoxyl amidated pectin and/or carrageenan; and
aii) one or more protein with an isoelectric point of 4.5 or greater; and/or one or more cationic mineral salt;
b) partially or fully dissolving the mixture;
c) ensuring the pH of the dissolved mixture is in the range 3.8 to 5.0;
d) boiling the mixture;
e) transferring the boiled mixture to moulds to set; and
f) conditioning the mixture to form a hydrogel composition in a unit dosage form.

This third general embodiment provides a method of manufacturing “gummies” with a high load of proteins and/or cationic mineral salts with acceptable taste and good stability.

Steps a) to f) are generally performed in alphabetical order (i.e., a) to f)) to ensure that the boiling step-which ensures gelation-occurs when the mixture is fully dissolved at the correct pH. However, a heavily buffered solution might not require the pH to be buffered or a pH step might occur after heating where less soluble ingredients are included.

In step a) the LM amidated pectin and/or carrageenan; as well as one or more protein with an isoelectric point of 4.5 or greater and/or one or more cationic mineral salt are added to water (or a water-based solution). Preferably, in step a) the LM amidated pectin and/or carrageenan of step ai) are first added to the water (preferably water warmed to a temperature for 40 to 80° C., more preferably 50 to 70° C.). Preferably, the LM amidated pectin and/or carrageenan are added in an amount sufficient to cause gelation after boiling and conditioning. Optionally, bulking agents are added as part of step ai). Preferably, the mixture of step ai) is dissolved prior to step aii). More preferably, the mixture of step ai) is heated to 70 to 90° C., more preferably 80 to 90° C., prior to step aii).

Preferably, in step a), the active ingredients (i.e., one or more protein with an isoelectric point of 4.5 or greater; and/or one or more cationic mineral salt) added as part of step aii) are added to the mixture after step ai). Optionally, bulking agents are added as part of step aii).

In step b) the mixture is allowed to partially or fully dissolve. This minimises unpredictable changes in pH. Preferably, step b) occurs at temperatures of 80 to 98° C. This ensures maximum dissolution without boiling. The dissolution time is not particularly limited and may preferably be for 1 to 20 minutes. Preferably, the mixture is allowed to fully dissolve. Preferably, the mixture of step b) is partially or fully dissolved by maintaining a temperature for 60 to 98° C. for 1 to 20 minutes.

In step c) the dissolved mixture is checked to ensure that the pH is in the range of 3.8 to 5.0. This may be performed by a standard pH probe. Preferably, the pH is in the range of 3.8 to 4.5, more preferably in the range of 4.0 to 4.5. Preferably, the pH is titrated to this range, more preferably using one or more organic acid. Preferred organic acids may be selected from the selected from the group consisting of ascorbic acid, citric acid, lactic acid, fumaric acid, tartaric acid, acetic acid, gluconic acid, and/or malic acid, more preferably citric acid, lactic acid and/or ascorbic acid even more preferably citric acid and/or lactic acid. Where a specific metal ion chelator is included (e.g., citrate or ascorbate) it may be preferably to use the equivalent organic acid (e.g., citric acid or ascorbic acid).

Preferably, step c) occurs after step b) and before step d). This ensures the optimal pH for gelation. However, the pH may be set within the range of 3.8 to 5.0 prior to dissolution (step b)) with a final pH adjustment after boiling the mixture (step d)). This is preferred for temperature sensitive active ingredients such as magnesium glycinate or calcium-d-pantothenate. Adding the active ingredients before boiling is preferred, especially for difficult-to-dissolve active ingredients that are temperature resistant such as magnesium citrate or calcium citrate.

In step d) the mixture is boiled. This ensures gelation and the dissolution of any undissolved active ingredients. Optionally, a final pH adjustment after boiling the mixture may be performed, especially where the active ingredient(s) has low solubility.

At the end of step d), the amounts of each component are preferably the same as in the hydrogel composition of the first embodiment. Preferably, the boiled mixture of step d) comprises 15 to 50 wt % of one or more protein with an isoelectric point of 4.5 or greater; and/or 8 to 50 wt % of one or more cationic mineral salt; and 1 to 5 wt % LM amidated pectin and/or carrageenan. Optionally, the boiled mixture of step d) comprises one or more organic acid in an amount of 0.1 to 20 wt %; one or more bulking agent in an amount of 20 to 80 wt %, and/or one or more fatty component in an amount of 0.1 to 10 wt %.

In step e) the boiled mixture is transferring to moulds to set. Preferably, the moulds are starch or silicone moulds. The size of the individual moulds in only limited by the requirement to form a unit dosage. Preferably, the weight of boiled mixture transferred to each mould is 0.5 to 9 g, preferably 1 to 4 g, more preferably 2 to 3 g.

In step f) the boiled mixture is conditioned to form a hydrogel composition in a unit dosage form. The conditioning allows the gel matrix to form and set as the boiled mixture cools. Preferably, the boiled mixture is conditioned for at least 4 hours, more preferably at least 12 hours, even more preferably about 24 hours. Longer conditioning steps are envisaged but are not required. When the hydrogel composition in a unit dosage form is removed from the mould, it is optionally coated with an oil and/or wax, preferably a vegetable oil and/or carnauba wax.

At the end of step f), the amounts of each component are preferably the same as in the hydrogel composition of the first embodiment. Preferably, the hydrogel composition in a unit dosage form of step f) comprises 15 to 50 wt % of one or more protein with an isoelectric point of 4.5 or greater; and/or 8 to 50 wt % of one or more cationic mineral salt; and 1 to 5 wt % low methoxyl amidated pectin and/or carrageenan. Optionally, the hydrogel composition in a unit dosage form of step f) comprises one or more organic acid in an amount of 0.1 to 20 wt %; one or more bulking agent in an amount of 20 to 80 wt %, and/or one or more fatty component in an amount of 0.1 to 10 wt %.

Optionally, one or more of the orally administrable hydrogel compositions of the method of manufacture described above are transferred to a shelf-stable packet or container. The packet or container is preferably selected from the group consisting of a tube, a tub, a pouch, a sachet, a bottle or a blister pack. The packet or container optionally includes instructions for administration.

In an embodiment, the invention relates to an orally administrable hydrogel composition in a unit dosage form obtained or obtainable by the method of manufacture described above.

### EXAMPLES

### Example 1—Preparation of Stable Gummies (Hydrogel Formulations) with Balanced Sourness

Example gummies (hydrogel compositions) with low methoxyl (LM) amidated pectin or carrageenan as the gelling agent were prepared as well as control gummies with high methoxyl (HM) pectin or gelatine. The gummies were tested for stability and taste, as described below. Attempts to produce high-load hydrogel formulations using agar and gellan gum gel, which are known to gel at mildly acidic pH values, failed. When the concentration of primary active ingredients was increased to ~8 wt % or greater, mouldable gels did not form when using agar and gellan gum gel.

### General Method of Production

The gelling agents (LM amidated pectin (Genu 102 LM AS-CAB from CP Kelco or Genu-106 AS YA from CP Kelco), carrageenan (Genugel GG-1435 from CP Kelco or Satiagel PG 500 from Cargill), HM pectin (Unipectine 569 or Unipectine 759 both from Cargill, Aglu Pectin HS-SB122 from JRS, Genu Pectin Vit conf from CP Kelco) or gelatine (Porcine Gelatine, 275 Bloom strength from Rousselot)) were immersed in water at 60° C., stirred, mixed with corn syrup (42 DE non-GMO 80% solids), inulin (Orafti® L95 from Beneo) or maltitol (Lycasin® 85/50, from Roquette), and heated to 80-95° C. to ensure dissolution. Pectins and carrageenan were added as 1:1 pectin:sugar mixtures. Next, the active ingredients (collagen (porcine or bovine collagen peptides from Gelita, e.g., VERISOL B (ovine skin) with a pI of 5.69 and average molecular weight of ~2 kDa), whey protein isolate (GermanProt 9000 WPI instantized with sunflower lecithin), pea protein isolate (Pea protein isolate PPI-20-B from LDC), calcium ascorbate, calcium citrate, magnesium glycinate or magnesium citrate) were added and the mixtures stirred for 1 minute. Next, food grade acid (ascorbic, citric, lactic, fumaric, tartaric and/or malic acid) was added to titrate the mixtures to the desired range (e.g., 3.8 to 5.0) and stirred for 5 minutes. As pectin can degrade above pH 4.5, higher pH values were not employed for pectin gelling agents. Sugar was added and mixed until dissolved. The General method of production temperature was maintained in the range of 80-90° C. for gelatine and 95-98° C. for pectins and carrageenan. Once the pH was in the correct range, the mixtures were boiled until enough water was evaporated to reach total solids in the range of 70-80 wt %. Next, the mixture was cooled to 80-85° C. for gelatine and 95-98° C. for pectins and carrageenan. Next, colourants, flavours (e.g., mixed berries) and (optionally) extra food grade acid was added and mixed until uniform. The pH of the final mixture was set within the range of 3.8-4.5 depending on the amount of active and the pH required for gelation, i.e. pH 3.8-4.5 for gelatine, pH 3.3-3.5 for HM pectin, pH 3.8-4.5 for LM amidated pectin and pH 4.0-4.5 for carrageenan. Next, the molten preparations were deposited in starch or silicone moulds and conditioned for 24 hours. Finally, the gummies (hydrogel formulations) were removed from the moulds and coated with a thin layer of oil and/or wax (for example vegetable oil and/or carnauba wax, of which Capol 42-3073 A MB and Capol 5108 are commercially available examples) to prevent sticking and optionally sugar coated. The final gummies (hydrogel formulations) were subjected to stability and taste testing as described below. As indicated above and below, not all control preparations formed gummies.

### Taste Testing

The gummies (hydrogel formulations) were offered to 7 volunteer panellists. Two gummies of about 3 grams were offered. The samples were coded with 3-digit numbers. The panellists were asked to evaluate for overall acceptance and sourness.

Gummies made with high level of primary active ingredients (e.g., ≥15 wt % protein or ≥8 wt % mineral concentration) and pH of 3.5 or less were found to have unacceptable sourness (intensely sour) and poor overall acceptance. These were samples made with HM pectin which only gels at pH values of 3.5 or less. The samples made with gelatine, LM amidated pectin or carrageenan with pH of 3.8 or higher had good overall acceptance and level of sourness (balanced sourness).

### Stability Testing

The gummies (hydrogel formulations) made with different gelling agents were stored at elevated temperatures of 45° C. and 68° C. in closed containers. The condition of the samples was monitored after 3 hours and 24 hours. After 3 hours all gelatine-based gummies had melted at both temperature conditions. The gummies made with either of the two pectin types or carrageenan retained their shape at both temperatures even after 24 hours.

### Analysis of Results

As summarised in the TABLE 1 to TABLE 7 (with all amounts in wt %) and discussion below, only the gummies (hydrogel formulations) formulated using LM amidated pectin or carrageenan could be produced as high-load hydrogel formulations with acceptable taste and good stability:

TABLE 1

| Formula type | Invention | Control | Control | Invention | Control | Control |
|---|---|---|---|---|---|---|
| LM Amidated Pectin | 2.8 | | | 2.8 | | |
| HM Pectin | | 2.8 | | | 2.8 | |
| Gelatine | | | 7 | | | 7 |
| Water | 20 | 20 | 20 | 20 | 20 | 20 |
| Sugar | 28.3 | 27.4 | 25.7 | 25.2 | 24.4 | 22.7 |
| Corn Syrup | 25.4 | 24.7 | 23.8 | 23.3 | 22.4 | 21.6 |
| Collagen peptides | 20 | 20 | 20 | 25 | 25 | 25 |
| Citric acid | 0.3 | 0.5 | 0.3 | 0.5 | 0.8 | 0.5 |
| Mixed berry flavour | 1 | 1 | 1 | 1 | 1 | 1 |
| Colour | 0.1 | 0.1 | 0.1 | 0.1 | 0.1 | 0.1 |
| Lactic Acid | 2.1 | 3.5 | 2.1 | 2.1 | 3.5 | 2.1 |
| pH | 4.0 | 3.5 | 4.1 | 4.1 | 3.5 | 4.2 |
| Balanced sourness | Yes | No | Yes | Yes | No | Yes |
| Stable at 68° C. | Yes | Yes | No | Yes | Yes | No |

TABLE 2

| Formula type | Invention | Control | Control | Invention | Control | Control |
|---|---|---|---|---|---|---|
| LM Amidated Pectin | 2.8 | | | 2.8 | | |
| HM Pectin | | 2.8 | | | 2.8 | |
| Gelatine | | | 8 | | | 8 |
| Water | 20 | 20 | 20 | 20 | 20 | 20 |
| Sugar | 28.0 | 27.2 | 25.5 | 25.5 | 24.7 | 23.0 |
| Corn Syrup | 30.7 | 29.9 | 28.0 | 28.0 | 27.1 | 25.3 |
| Magnesium Citrate | 15 | 15 | 15 | 20 | 20 | 20 |
| Citric acid | 0.3 | 0.5 | 0.3 | 0.5 | 0.8 | 0.5 |
| Mixed berry flavour | 1 | 1 | 1 | 1 | 1 | 1 |
| Colour | 0.1 | 0.1 | 0.1 | 0.1 | 0.1 | 0.1 |
| Lactic Acid | 2.1 | 3.5 | 2.1 | 2.1 | 3.5 | 2.1 |
| pH | 4.0 | 3.5 | 4.1 | 4.1 | 3.5 | 4.2 |
| Balanced sourness | Yes | No | Yes | Yes | No | Yes |
| Stable at 68° C. | Yes | Yes | No | Yes | Yes | No |

TABLE 3

| Formula type | Invention | Control | Control | Invention | Control | Control |
|---|---|---|---|---|---|---|
| LM Amidated Pectin | 2.8 | | | 2.8 | | |
| HM Pectin | | 2.8 | | | 2.8 | |
| Gelatine | | | 8 | | | 8 |
| Water | 20 | 20 | 20 | 20 | 20 | 20 |
| Sugar | 28.0 | 27.2 | 25.5 | 25.5 | 24.7 | 23.0 |
| Corn Syrup | 22.2 | 21.7 | 19.7 | 19.4 | 21.3 | 16.7 |
| Magnesium Glycinate | 15 | 15 | 15 | 20 | 20 | 20 |
| Citric acid | 13.5 | 14.5 | 13.5 | 18.0 | 19.5 | 18.0 |
| Mixed berry flavour | 1 | 1 | 1 | 1 | 1 | 1 |
| Colour | 0.1 | 0.1 | 0.1 | 0.1 | 0.1 | 0.1 |
| Lactic Acid | 1 | 1 | 1 | 1 | 1 | 1 |
| pH | 4.0 | 3.5 | 4.1 | 4.0 | 3.5 | 4.1 |
| Balanced sourness | Yes | No | No* | Yes | No | No* |
| Stable at 68° C. | Yes | Yes | No* | Yes | Yes | No* |

*The gelatine precipitated out of solution, so gummies could not be made

TABLE 4

| Formula type | Invention | Control | Control | Invention | Control | Control |
|---|---|---|---|---|---|---|
| Carrageenan | 2.5 | | | 2.5 | | |
| HM Pectin | | 2.8 | | | 2.8 | |
| Gelatine | | | 7 | | | 7 |
| Water | 20 | 20 | 20 | 20 | 20 | 20 |
| Sugar | 26.4 | 25.2 | 24.2 | 21 | 19 | 18.7 |
| Corn Syrup | 28 | 27.4 | 25.7 | 22.9 | 22.6 | 20.7 |
| Collagen peptides | 20 | 20 | 20 | 30 | 30 | 30 |
| Citric acid | 2 | 3.5 | 2 | 2.5 | 4.5 | 2.5 |
| Mixed berry flavour | 1 | 1 | 1 | 1 | 1 | 1 |
| Colour | 0.1 | 0.1 | 0.1 | 0.1 | 0.1 | 0.1 |
| Lactic Acid | | | | | | |
| pH | 4 | 3.4 | 4.1 | 4 | 3.3 | 4.1 |
| Balanced sourness | Yes | No | Yes | Yes | No | Yes |
| Stable at 68° C. | Yes | Yes | No | Yes | Yes | No |

TABLE 5

| Formula type | Invention | Control | Control | Invention | Control | Control |
|---|---|---|---|---|---|---|
| Carrageenan | 1.9 | | | 2.2 | | |
| HM Pectin | | 2.8 | | | 2.8 | |
| Gelatine | | | 8 | | | 8 |
| Water | 20 | 20 | 20 | 20 | 20 | 20 |
| Sugar | 33.0 | 32.4 | 30.0 | 24.3 | 23.4 | 22.0 |
| Corn Syrup | 35.7 | 34.2 | 32.6 | 26.8 | 25.0 | 23.3 |
| Calcium citrate | 8 | 8 | 8 | 25 | 25 | 25 |
| Citric acid | 0.3 | 1.5 | 0.3 | 0.6 | 2.7 | 0.6 |
| Mixed berry flavour | 1 | 1 | 1 | 1 | 1 | 1 |
| Colour | 0.1 | 0.1 | 0.1 | 0.1 | 0.1 | 0.1 |
| Lactic Acid | | | | | | |
| pH | 4 | * | 4.1 | 4 | * | 4.1 |
| Balanced sourness | Yes | No* | Yes | Yes | No* | Yes |
| Stable at 68° C. | Yes | No* | No | Yes | No* | No |

*Gummies could not be formed the bulk set as hard gel when calcium citrate was added

TABLE 6

| Formula type | Invention | Control | Control | Invention | Control | Control |
|---|---|---|---|---|---|---|
| Carrageenan | 2.8 | | | 2.8 | | |
| HM Pectin | | 2.8 | | | | |
| Gelatine | | | 8 | | | 8 |
| Water | 20 | 20 | 20 | 20 | | 20 |
| Sugar | 27.3 | 25.0 | 24.4 | 19.5 | | 18.2 |
| Corn Syrup | 29.9 | 28.6 | 27.6 | 23.6 | | 19.7 |
| Calcium Ascorbate | 8 | 8 | 8 | 14 | | 14 |
| Citric acid | 10.9 | 14.5 | 10.9 | 19 | | 19 |
| Mixed berry flavour | 1 | 1 | 1 | 1 | | 1 |
| Colour | 0.1 | 0.1 | 0.1 | 0.1 | | 0.1 |
| Lactic Acid | | | | | | |
| pH | 4 | * | 4.1 | 4 | | 4.1 |
| Balanced sourness | Yes | No* | No** | Yes | | No** |
| Stable at 68° C. | Yes | No* | No** | Yes | | No** |

*Gummies could not be formed the bulk set as hard gel when calcium ascorbate was added
**Gummies were very soft and sticky so could not hold their shape

TABLE 7

| Formula type | Invention | Control | Control | Invention | Control | Control |
|---|---|---|---|---|---|---|
| LM Amidated Pectin | 2.4 | | | 2.4 | | |
| HM Pectin | | 2.4 | | | 2.4 | |
| Gelatine | | | 5 | | | 5 |
| Water | 25 | 25 | 25 | 25 | 25 | 25 |
| Sugar | 26.3 | 24.2 | 23.8 | 24.4 | 22.5 | 22.1 |
| Corn Syrup | 27.9 | 26.2 | 25.4 | 27.1 | 24.7 | 24.4 |
| Whey protein Isolate | 15 | 15 | 15 | | | |
| Pea Protein | | | | 20 | 20 | 20 |
| Citric acid | 4.7 | 6.1 | 4.7 | 2.4 | 4.3 | 2.4 |
| Mixed berry flavour | 1 | 1 | 1 | 1 | 1 | 1 |
| Colour | 0.1 | 0.1 | 0.1 | 0.1 | 0.1 | 0.1 |
| Lactic Acid | | | | | | |
| pH | 4 | 3.5 | 4.1 | 4 | 3.5 | 4.1 |
| Balanced sourness | Yes | No | Yes | Yes | No | Yes |
| Stable at 68° C. | Yes | Yes | No | Yes | Yes | No |

TABLE 1 to TABLE 3 confirm that LM amidated pectin produces palatable gummies with balanced sourness and excellent stability whereas those made with HM pectin or gelatine are either intensely sour (HM Pectin) or unstable (gelatine). The results confirm that palatable gummies with balanced sourness and excellent stability can be produces with high amounts of protein (e.g., 20 or 25 wt % collagen peptides) or minerals (e.g., 15-20 wt % magnesium salts of different types).

TABLE 4 to TABLE 6 confirm that carrageenan also produces palatable gummies with balanced sourness and excellent stability. The results confirm that the palatable gummies with balanced sourness and excellent stability can be produces with high amounts of protein (e.g., 20 or 30 wt % collagen peptides) or minerals (e.g., 8-25 wt % calcium salts of different types).

TABLE 7 confirms that palatable gummies with balanced sourness and excellent stability can be produces with high amounts of other proteins with an isoelectric point of 4.5 or greater (e.g., intact whey protein (pI 4.6-5.2) and intact pea protein isolate (pI~4.5)).

The data confirm that gummies (hydrogel formulations) with high amounts of protein (e.g., animal proteins, dairy proteins and/or plant proteins) and minerals (e.g., calcium and/or magnesium) can be produced at a pH of 3.8-5.0 when low methoxyl (LM) amidated pectin and/or carrageenan are used as gelling agents. In particular, cationic mineral salts and proteins with an isoelectric point (pI) of 4.5 or greater (i.e., that maintain some positively charge close to and below the pI) form stable, palatable “gummy” formulations in the presence of pectin and/or carrageenan.

To confirm the tolerability of the disclosed gummies to different bulking agents, two further hydrogel compositions were produced with the compositions shown in

TABLE 8
(with all amounts in wt %):

| Formula type | Invention | | Invention |
|---|---|---|---|
| Carrageenan | 3 | Amidated Pectin | 2.8 |
| Water | 17 | Water | 17 |
| Collagen Peptides | 25 | Magnesium Citrate | 15 |
| Inulin | 54.2 | Maltitol solution | 64.4 |
| Lactic acid | 2.2 | Citric acid | 2 |
| Flavor | 1 | Flavor | 1 |
| Monk Fruit Extract | 0.5 | Monk Fruit Extract | 0.5 |
| Color | 0.1 | Color | 0.1 |
| | | Lactic acid | 2.2 |
| pH | 4.4 | pH | 4 |
| Balanced sourness | Not tested | | Not tested |
| Stable at 68° C. | Yes | | Yes |

TABLE 8 confirms that gummies with excellent stability can be produced with fibre (inulin) and sugar alcohols (maltitol) as bulking agents.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

1: An orally administrable hydrogel composition in a unit dosage form, comprising:
15 to 50 wt % of one or more protein with an isoelectric point of 4.5 or greater; and/or
8 to 50 wt % of one or more cationic mineral salt; and
1 to 5 wt % carrageenan and/or low methoxyl amidated pectin;
wherein the composition has a pH in the range of 3.8 to 5.0.

2: The hydrogel composition according to claim 1, wherein the one or more protein with an isoelectric point of 4.5 or greater is selected from the group consisting of:
an animal protein or a hydrolysed version thereof;
a dairy protein selected from the group consisting of a casein protein, a whey protein, a hydrolysed version thereof, and any combination thereof, and
a plant protein or a hydrolysed version thereof.

3: The hydrogel composition according to claim 1, wherein the one or more cationic mineral salt is at least one of a calcium salt or a magnesium salt.

4: The hydrogel composition according to claim 1, wherein:
the one or more protein with an isoelectric point of 4.5 or greater is selected from the group consisting of collagen, whey protein, pea protein, a hydrolysed version thereof, and any combination thereof; and/or
the one or more cationic mineral salt is selected from the group consisting of calcium ascorbate, calcium citrate, magnesium glycinate and magnesium citrate.

5: The hydrogel composition according to claim 1, further comprising one or more organic acids in an amount of 0.1 to 20 wt %.

6: The hydrogel composition according to claim 1, further comprising one or more bulking agent in an amount of 20 to 80 wt %.

7: The hydrogel composition according to claim 1, further comprising one or more fatty component selected from the group consisting of a fat, a diglyceride, a monoglyceride and an emulsifier, in an amount of 0.1 to 10 wt %.

8: The hydrogel composition according to claim 1, further comprising one or more additional vitamin, mineral or micronutrient.

9: The hydrogel composition according to claim 1, wherein the one or more protein with an isoelectric point of 4.5 or greater is present in an amount of 15 to 40 wt %.

10: The hydrogel composition according to claim 1, wherein the one or more cationic mineral salt is present in an amount of 8 to 40 wt %.

11: The hydrogel composition according to claim 1, wherein the carrageenan and/or low methoxyl amidated pectin is present in an amount of 1 to 3 wt %.

12: The hydrogel composition according to claim 1, wherein the composition has a pH in the range of 3.8 to 4.5.

13: The hydrogel composition according to claim 7, wherein the one or more fatty component selected from the group consisting of a fat, a diglyceride, a monoglyceride and/or an emulsifier is present in an amount of 1 to 9 wt %.

14: The hydrogel composition according to claim 1, wherein the composition is substantially free or free from a fat, a diglyceride, a monoglyceride and/or an emulsifier.

15: The hydrogel composition according to claim 1, wherein the composition comprises the one or more protein with an isoelectric point of 4.5 or greater and the one or more cationic mineral salt in a total amount of 23 to 87 wt %.

16: The hydrogel composition according to claim 1, wherein the unit dosage form has a weight of 0.5 g to 9 g.

17: The hydrogel composition according to claim 1, wherein the composition is stable at 68° C. in a closed container for 3 hours.

18: A shelf-stable packet or container comprising one or more of the orally administrable hydrogel compositions according to claim 1.

19: A method of manufacturing an orally administrable hydrogel composition in a unit dosage form, the method comprising:
a) adding to water to form a mixture:
ai) carrageenan and/or low methoxyl amidated pectin; and
aii) one or more protein with an isoelectric point of 4.5 or greater; and/or one or more cationic mineral salt;
b) partially or fully dissolving the mixture;
c) ensuring the pH of the dissolved mixture is in the range 3.8 to 5.0;
d) boiling the mixture;
e) transferring the boiled mixture to moulds to set; and
f) conditioning the mixture to form a hydrogel composition in a unit dosage form.

20: An orally administrable hydrogel composition in a unit dosage form obtained or obtainable by the method according to claim 19.
